(19) United States
(12) Patent Application Publication
(10) Pub. No.: US 2023/0219999 A1
(43) Pub. Date: Jul. 13, 2023

(54) METHOD FOR PRODUCING PEPTIDES OR PROTEINS OR PEPTIDOMIMETICS

(71) Applicant: STRAINCHEM, Saint-Beauzire (FR)

(72) Inventors: Jean-Jacques YOUTE TENDOUNG, Pont du Château (FR); Audrey SERRE, Clermont-Ferrand (FR)

(73) Assignee: STRAINCHEM, Saint-Beauzire (FR)

(21) Appl. No.: 17/605,008

(22) PCT Filed: May 4, 2020
(86) PCT No.: PCT/FR2020/000158

§ 371 Date: Oct. 20, 2021

(30) Foreign Application Priority Data
May 2, 2019 (FR) 1904604

(51) Int. Cl. C07K 1/04; C07K 1/06

(52) U.S. Cl. CPC C07K 1/042; C07K 1/062; C07K 1/063

(57) ABSTRACT

A process for synthesizing peptides or proteins or peptidomimetics by successive elongation, with units, of the second end (primary or secondary amine function, hydroxyl function or thiol function) of a peptide or protein or peptidomimetic chain, characterized in that: said units are selected from the group made up of: α, β or γ-amino acids, α, β or γ-hydroxy acids and α, β or γ-mercapto acids (natural or unnatural or synthetic), the molecules having at least two functional groups; —the first end of said peptide or protein or peptidomimetic is bonded by a covalent bond to an anchoring molecule that is soluble in organic solvents such as halogenated solvents (methylene chloride, chloroform), ethyl acetate, tetrahydrofuran, 2-methyltetrahydrofuran, isooctane, cyclohexane, hexane(s), methylcyclohexane or methyl tert-butyl ether, or aromatic solvents such as benzene or toluene, or any other suitable solvent.

### TECHNICAL FIELD OF THE INVENTION

The present invention relates to the chemistry of peptides or proteins or peptidomimetics, and more particularly their chemical syntheses from bifunctional molecules, and in particular α, β or γ-amino acids and/or α, β or γ-hydroxy acids and/or α, β or γ-mercapto acids.

More specifically, the invention relates to a method for producing peptides or proteins or peptidomimetics in solution. This method does not use conventional protection groups such as tert-butoxycarbonyl (Boc) or fluorenylmethoxycarbonyl (Fmoc) on the amine function of α, β, or γ-amino acid. Likewise, it is not necessary to use protection groups on the hydroxyl function of α, β, or γ-hydroxy acid, or on the thiol function of α, β, or γ-mercapto acid.

This method is based on the use of activated α, β, or γ-amino acids or α, β, or γ-hydroxy acids or α, β, or γ-mercapto acids respectively in the form of 2,2-bis(trifluoromethyl)-1,3-oxazolidin-5-one or 2,2-bis(trifluoromethyl)-1,3-oxazinan-6-one, or 2,2-bis(trifluoromethyl)-1,3-oxazepan-7-one or 2,2-bis(trifluoromethyl)-1,3-dioxolan-4-one, or 2,2-bis(trifluoromethyl)-1,3-dioxan-4-one, or 2,2-bis(trifluoromethyl)-1,3-dioxepan-4-one, or 2,2-bis(trifluoromethyl)-1,3-oxathiolan-5-one, or 2,2-bis(trifluoromethyl)-1,3-oxathian-6-one, or 2,2-bis(trifluoromethyl)-1,3-oxathiepan-7-one or derivatives thereof and of a family of anchoring molecules, namely derivatives of polyolefins or polyolefin oligomers or polyalkenes. The anchoring molecule is bonded to a molecule having at least two electrophilic and/or nucleophilic functional groups, and in particular to a first α, β, or γ-amino acid or α, β, or γ-hydroxy acid, or α, β, or γ-mercapto acid, which will then be the subject of successive elongation/iteration steps to lead to peptides or proteins or peptidomimetics.

This method allows to obtain peptides or proteins or peptidomimetics in a more efficient (that is to say with a reduced number of steps), faster way, which are purer or easier to purify than the current methods on solid support or in solution. It is easy to automate.

### PRIOR ART

The remarkable growth in the development of therapeutic peptides or proteins or peptidomimetics over the last decade has led to a large number of approvals of new molecules on the market (see the publications of J. Med. Chem., 2018, 22, 1382-1414, Bioorg. Med. Chem., 2018, 26, 2700-2707); thus therapy based on peptides or proteins or peptidomimetics has become one of the most dynamic segments in the pharmaceutical industry. Cancers, metabolic diseases and diseases of the central nervous system are the main therapeutic areas accelerating the demand for new peptides or proteins or therapeutic peptidomimetics.

However, a number of obstacles prevent the widespread adoption of peptides, or proteins or peptidomimetics in therapy. Mention may be made, for example, of their short metabolic half-life and their hydrophilic nature. Another key factor, and by far the most important, retarding the growth of therapeutic applications of peptides or proteins or peptidomimetics is their mode of production.

The very first liquid-phase peptide synthesis was performed over a century ago by E. Fisher and E. Fourneau (Ber. Dtsch. Chem. Ges. 1901, 34, 2868-2879). Since then, many chemists have made improvements; this is the case with Bodansky and du Vigneaud (J. Am. Chem. Soc., 1959, 51, 5688-5691), with Beyerman and al. (Rec. Trav. Chim., Netherlands 1973, 92, 481-492), R. K. Sharma and R. Jain (Synlett 2007, 603-606), Nodal and al. (Nature Chemistry 2017, 9, 571-577), Liu and al. (Org. Lett., 2018, 20, 612-615), and with Muramatsu and al. (ACS Catal., 2018, 8, 2181-2187).

The most frequently used peptide or protein or peptidomimetic synthesis routes involve temporary protection of the amine function (Nα) of amino acids. Today, the main protection groups used are the tert-butoxycarbonyl group, this approach is commonly called the “Boc” strategy, and the fluorenylmethoxycarbonyl group, this approach is commonly called the “Fmoc” strategy. These two peptide synthesis routes are known to the person skilled in the art (see Section 7-5 of the “Biochemistry” manual by D. Voet and J. G. Voet, 2^{nd} edition, Brussels 2005). In practice, the amino acids are supplied in the protected state on the amine function (Nα) by the Fmoc or Boc group, and are directly involved in the activation/coupling reactions.

The amino acids can be used in the liquid phase or on a solid support; in the latter case, the amino acid protected on the amine function (Nα) is attached to a resin insoluble in organic solvents, this is the synthesis of Merrifield (J. Am. Chem. Soc., 1963, 85, 2149-2154). This is a well-controlled method, which however has some disadvantages such as: the cost of reagents used in excess and the lack of homogeneity of the peptides synthesized. The system is said to be degenerate, which generates additional costs for purifications by preparative high performance liquid chromatography.

In Liquid Phase Peptide Synthesis (LPPS), all reactions take place in homogeneous solution. This methodology was described by Bodansky and du Vigneaud (J. Am. Chem. Soc., 1959, 51, 5688-5691). The carboxylic acid function (C-terminal) of the starting amino acid is protected in the form of a methyl ester, and the following amino acids are condensed successively after the protection of their amine function (Nα) by a carboxybenzyl group (abbreviated Cbz) followed by the activation of their carboxylic acid function (C-terminal) by a nitrophenyl ester. All synthetic intermediates are purified by precipitation or washing with water (extraction). This peptide synthesis methodology is long, tedious and generates peptides with low yields. By way of example, mention may be made of the synthesis of ACTH with an overall yield of about 7%, described by Schwyzer and Sieber (Helv. Chim. Acta 1966, 49, 134-158).

A modification of this methodology has been reported by Beyerman and al. (Rec. Trav. Chim. Netherlands 1973, 92, 481-492). It consists in protecting the carboxylic acid function (C-terminal) of an amino acid or of a peptide in the form of a benzyl ester and carrying out the coupling (or condensation) reaction in the presence of an excess of Na-protected amino acid anhydride, with a view to improving yields. Finally, although the yields of the coupling reactions are increased, there is a loss of solubility of the peptide in the organic phase when the latter reaches about five amino acids.

Other strategies allowing the solubilization of amino acids, in order to facilitate peptide synthesis, have been developed. Mention may be made of the work of Narita (Bull. Chem. Soc. Jap., 1978, 51, 1477-1480), the work of Bayer and Mutter (Nature 1972, 237, 512-513) on polyethylene glycol as a solubilization adjuvant, and patents EP 0 017 536 (Sanofi) and EP 2 612 845 A1 and US 2014/0296483 (Ajinomoto Co., Inc.) on solubilizing anchoring molecules.

Peptide synthesis strategies are also known which use bifunctional groups, that is to say groups which are capable simultaneously of activating the carboxylic acid function (C-terminal) and protecting the amine function (Nα) of the amino acid, forming highly reactive intermediate ring structures. This is the case with N-carboxyanhydrides (abbreviated NCAs) (see Ber. Dtsch. Chem. Ges., 1906, 39, 857-861; Ber. Dtsch. Chem. Ges., 1907, 40, 3235-3249; Ber. Dtsch. Chem. Ges., 1908, 41, 1721-172; J. Am. Chem. Soc., 1957, 79, 2153-2159; J. Am. Chem. Soc., 1947, 69, 1551-1552). Reactive intermediates were synthesized from amino acids and dichlorodimethylsilane derivatives (see S. H. van Leeuwen and al., Tetrahedron Letters 2002, 43, 9203-9207 and WO 00/37484 A1). Amino acid derivatives activated by boron trifluoride etherate were invented for the synthesis of peptides (see S. H. van Leeuwen and al., Tetrahedron Letters 2005, 46, 653-3656). The activation of amino acids in the presence of hexafluoroacetone has also been described (see Chem. Ztg., 1990, 114, 249-251 and J. Spengler and al., Chem. Rev., 2006, 106, 4728-4746).

All these methods for synthesizing peptides or proteins or peptidomimetics, in solution or on a solid support, have at least one or more disadvantages among the following: the use of protection groups, the use of reagents in excess, the possibility of racemization, the low solubility of the peptides during synthesis in organic solvents, the limitation of the size of the peptide, expensive and polluting purifications, complex experimental protocols or the possibility of polymerization. In general, the examination of the abundant literature available in the field of peptide synthesis shows that difficulties persist to produce peptides or proteins or peptidomimetics of high purity, with good yields, at low cost and with a lower ecological footprint.

The problem that the present application proposes to solve is the design of a new methodology for the synthesis of peptides or proteins or peptidomimetics allowing to remove the obstacles related to their access or production left in the prior art.

### OBJECT OF THE INVENTION

According to the invention, the problem is solved by a method for synthesizing peptides or proteins or peptidomimetics in liquid phase which includes the combination of two essential features which are detailed below.

The first object of the present invention is a method for synthesizing peptides or proteins or peptidomimetics by successive elongation of the second end of a Q^{a}-E-Q^{b} type molecule, where Q^{a} and Q^{b} can be identical or different and represent an electrophile and/or nucleophile function, and E represents a spacer. Said second end can in particular be a primary or secondary amine, a hydroxyl or a thiol, of an α, β, γ or δ-amino acid or α, β, γ or 5-hydroxy acid or α, β, γ or δ-mercapto acid or peptide or protein or peptidomimetic, characterized in that said units are selected from the group made up of: (natural or unnatural or synthetic) α, β, γ or δ-amino acids or α, β, γ or δ-hydroxy acids or α, β, γ or δ-mercapto acids. In addition, the first end of said Q^{a}-E-Q^{b} type molecule (for example of said α, β, γ or δ-amino acid or α, β, γ or δ-hydroxy acid or α, β, γ or δ-mercapto acid) or of said peptide or protein or peptidomimetic is attached to an anchoring molecule soluble in organic solvents such as halogenated solvents (methylene chloride, chloroform), ethyl acetate, tetrahydrofuran, 2-methyletetrahydrofuran, isooctane, cyclohexane, hexane(s), methylcyclohexane, methyl tert-butyl ether or aromatic solvents such as benzene or toluene or any other suitable solvent.

The first essential feature is the use of a family of specific anchoring molecules. According to the invention, the anchoring molecules are polyolefins or polyolefin oligomers or polyalkenes. The method according to the invention provides access to high purity (natural or unnatural or synthetic) peptides or proteins or peptidomimetics. This method generates savings of steps and atoms due to the absence of the use of protection groups (on the amine, hydroxyl or thiol functions of the main chain) and of coupling agents and therefore, financial savings. In the end, the method is more respectful of the environment.

The second essential feature is the use of bifunctional Q^{a}-E-Q^{b} type molecules wherein the groups Q^{a} and Q^{b} may be the same or different, and are selected from the electrophilic groups and/or the nucleophilic groups, and E represents a spacer. Advantageously, Q^{a} and Q^{b} are selected from the group made up of chemical functions such as: alcohols, aldehydes, primary amines, secondary amines, azides, ethynils, halogens, thiols, vinyls, and/or the spacer E is selected from the group made up of structural units such as: aromatic, heteroaromatic, saturated alkyl chains (branched or not), unsaturated alkyl chains (branched or not), glycols (and preferably polyethylene glycol).

In the advantageous case where use is made, as a bifunctional molecule, of Q^{a}-E-Q^{b} type molecule, an α, β or γ-amino acid or an α, β or γ-hydroxy acid or an α, β or γ-mercapto acid, these compounds are used in their activated forms, namely 2,2-bis(trifluoromethyl)-1,3-oxazolidin-5-one or 2,2-bis(trifluoromethyl)-1,3-oxazinan-6-one or 2,2-bis(trifluoromethyl)-1,3-oxazepan-7-one or 2,2-bis(trifluoromethyl)-1,3-dioxolan-4-ones or 2,2-bis(trifluoromethyl)-1,3-dioxan-4-one or 2,2-bis(trifluoromethyl)-1,3-dioxepan-4-one or 2,2-bis(trifluoromethyl)-1,3-oxathiolan-5-one or 2,2-bis(trifluoromethyl)-1,3-oxathian-6-one or 2,2-bis(trifluoromethyl)-1,3-oxathiepan-7-one or derivatives thereof.

Diagram 1 shows the structure of these activated forms. The latter are prepared from the corresponding α, β or γ-amino acids (this expression meaning here: α-amino acids, β-amino acids or γ-amino acids), or α, β or γ-hydroxy acids (this expression meaning here: α-hydroxy acids, β-hydroxy acids or γ-hydroxy acids), or α, β or γ-mercapto acids (this expression meaning here: α-mercapto acids, β-mercapto acids or γ-mercapto acids).

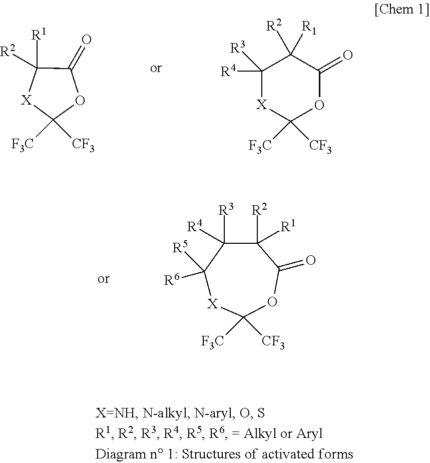

To date, the possibility of easily preparing in solution, peptides consisting of more than four amino acids which are different or not, using activated amino acid derivatives in the form of 2,2-bis(trifluoromethyl)-1,3-oxazolidin-5-one, has never been demonstrated.

It is precisely the object of the invention which proposes the use of activated acids in the form of 2,2-bis(trifluoromethyl)-1,3-oxazolidin-5-one or 2,2-bis(trifluoromethyl)-1,3-oxazinan-6-one or 2,2-bis(trifluoromethyl)-1,3-oxazepan-7-one or 2,2-bis(trifluoromethyl)-1,3-dioxolan-4-ones or 2,2-bis(trifluoromethyl)-1,3-dioxan-4-one or 2,2-bis(trifluoromethyl)-1,3-dioxepan-4-one or 2,2-bis(trifluoromethyl)-1,3-oxathiolan-5-one or 2,2-bis(trifluoromethyl)-1,3-oxathian-6-one or 2,2-bis(trifluoromethyl)-1,3-oxathiepan-7-one or derivatives thereof in the presence of an anchoring molecule allowing the production of peptides or proteins or peptidomimetics of high purity, in liquid (or solution) phase.

The method for synthesizing peptides or proteins or peptidomimetics according to the invention proceeds by successive elongation of the second end (primary or secondary amine, hydroxyl or thiol) of a peptide or protein or peptidomimetic chain whose first end is attached to a molecule anchor soluble in an organic solvent. Said anchoring molecule includes a polyolefin chain or polyolefin or polyalkene oligomers having at least 10 monomer units, and preferably between 15 and 350 monomer units.

In an advantageous embodiment, said polyolefin chain is a polyisobutene (PIB) chain. In particular, said anchoring molecule can be a polyolefin. The polyolefin chain can be functionalized at least at one of its ends. Alternatively, the polyolefin chain or polyolefin or polyalkene oligomer may comprise a number of unsaturated carbon-carbon bonds not exceeding 5%, and preferably not exceeding 3%, and/or the anchoring molecule can have a weight average molecular weight comprised between 600 and 20000, and preferably between 700 and 15000.

In a particular embodiment, said anchoring molecule includes a polyolefin chain (or is a polyolefin chain) terminated by at least one group selected from the group made up of:
a function —X^{a}, where X^{a} is selected from the group made up of: —OH, —NH₂, —NHR^{a} (R^{a}=alkyl or aryl), —SH;

a function —Y—C₆H₄X^{b}, where
Y is O, S, CH₂ or absent,
X^{b} is selected from the group made up of: —OH, —NH₂, —NHR^{a}, —SH, —CX^{a}R^{a}R^{b}, —C₆H₃R^{c}(CR^{a}X^{a}),

where: R^{b} is selected from the group made up of —H, -Aryl, —Heteroaryl, -Alkyl, and

R^{c} is selected from the group made up of —H, -Alkyl, —O-Alkyl, -Aryl, —O-Aryl, —Heteroaryl, —O-Heteroaryl;
a function —CR^{d}═CH—CHX^{a} or a function —CR^{d}H—CH═CH—CHX^{a}, where X^{a} has the meaning defined above, and R^{d} is methyl or ethyl.

In particular, X^{a} can be a primary or secondary amine function, an alcohol, a thiol or a phenol.

In an advantageous embodiment, the weight average molecular weight of the anchoring molecules, apart from the terminal functionalization (for example —X^{a}, —Z—C₆H₄X^{b} or —CR^{d}═CH—CHX^{a} as defined above), is comprised between 600 and 20000, and preferably between 700 and 15000. Above a weight average molecular weight of approximately 20000, these molecules may have a too great viscosity, which would risk limiting their solubility in organic solvents used for the coupling/elongation or iteration step.

Some PIB derivatives used in the context of the present invention are commercially available, as ligands for homogeneous catalysis. By way of example, use can be made of 2-methyl-3-[polyisobutyl(12)]propanol (weight average molecular weight 757, including terminal functionalization) or 4-[polyisobutyl(18)]phenol (weight average molecular weight 1104, including terminal functionalization) which are distributed, respectively, under the references 06-1037 and 06-1048 by the company Strem Chemicals. These two molecules are polyisobutenes derivatives whose chain is terminated, respectively, by a group —CH₂—C(CH₃)(H)—CH₂—OH (that is to say isopropanol) and by a group —CH₂—C(CH₃)₂—C₆H₄—OH (that is to say phenol).

According to one feature of the invention, the use of an anchoring molecule soluble in an organic solvent as described above (and more particularly the use of polyolefins), is capable also of acting as a liquid carrier or a protection group of the carboxylic acid function (C-terminal) or of any other chemical function (side chain(s)) of an α, β or γ-amino acid or α, β or γ-hydroxy acid or α, β or γ-mercapto acid, or any other molecule having at least two functional groups. It also allows the solubilization of the anchored peptides or proteins or peptidomimetics and their syntheses in organic solution (halogenated and/or non-halogenated solvents).

According to another feature of the invention, the use of an anchoring molecule soluble in an organic solvent as described above (and more particularly the use of polyolefins), and insoluble in some polar solvents (such as water and/or ethanol and/or acetonitrile), facilitates the purification of α, β or γ-amino acids or α, β or γ-hydroxy acids or α, β or γ-mercapto acids or peptides or proteins or peptidomimetics anchored by simple extraction (washing) or simple filtration on silica. Thus, a simple extraction or a simple filtration allows to obtain the anchored peptides or proteins or peptidomimetics with high chemical purity.

According to yet another feature of the invention, use is made of commercially available anchoring molecules, or anchoring molecules which can be synthesized simply and directly from commercially available precursors, in particular some polyisobutene (PIB) derivatives.

According to yet another feature of the invention, the α, β or γ-amino acids or α, β or γ-hydroxy acids or α, β or γ-mercapto acids are reacted respectively in their activated forms 2,2-bis(trifluoromethyl)-1,3-oxazolidin-5-one or 2,2-bis(trifluoromethyl)-1,3-dioxolan-4-one or 2,2-bis(trifluoromethyl)-1,3-oxathiolan-5-one or (2,2-bis(trifluoromethyl)-1,3-oxazinan-6-one, 2,2-bis(trifluoromethyl)-1,3-dioxan-4-one, 2,2-bis(trifluoromethyl)-1,3-oxathian-6-one, 2,2-bis(trifluoromethyl)-1,3-oxazepan-7-one, 2,2-bis(trifluoromethyl)-1,3-dioxepan-4-one, 2,2-bis(trifluoromethyl)-1,3-oxathiepan-7-one and derivatives thereof, in the presence of the anchoring molecule in an appropriate solvent (or a mixture of solvents), at a temperature comprised between −20° C. and 150° C. In one embodiment, the reaction is carried out in any inert liquid solvent (or mixture) capable of dissolving the reagents. Applicable solvents comprise, without limitation, halogenated or non-halogenated hydrocarbons. The preferred solvents are tetrahydrofuran, ethyl acetate, 2-methyltetrahydrofuran, propylene carbonate, or any other solvent or mixture of solvents capable of dissolving these two chemical species.

According to yet another feature of the invention, the reactions between the PIB derivatives and the activated α, β or γ-amino acids or activated α, β or γ-hydroxy acids or activated α, β or γ-mercapto acids are carried out in batch chemistry (in particular in a flask or cistern), but preferably, they are carried out in flow chemistry (also called continuous flow chemistry).

According to yet another feature of the invention, the α, β or γ-amino acids or α, β or γ-hydroxy acids or α, β or γ-mercapto acids having side chains which are incompatible with the conditions of the reaction of anchoring/elongation or iteration, can be temporarily masked by an appropriate protection group. It can in particular be selected from the group made up of:
tert-butoxycarbonyl (abbreviated Boc),
fluorenylmethoxycarbonyl (abbreviated Fmoc),
benzyl (abbreviated Bzl),
trityl (abbreviated Trt),
carboxybenzyl (abbreviated Cbz),
2,2,4,6,7-pentamethyldihydrobenzofuran-5-sulfonyl (abbreviated Pbf), 4-methoxy-2,3,6-trimethylbenzenesulphonyl (abbreviated Mtr).

It is also possible to use any other protection group compatible with the present method.

According to yet another feature of the invention, said anchoring molecule reacts with a first activated α, β or γ-amino acid or α, β or γ-hydroxy acid or α, β or γ-mercapto acid (here abbreviated AAA1), leading to a covalent bond between α, β or γ-amino acid or α, β or γ-hydroxy acid or α, β or γ-mercapto acid and the anchoring molecule.

According to yet another feature of the invention, said peptide or protein or peptidomimetic chain is formed of n units of α, β or γ-amino acids or α, β or γ-hydroxy acids or α, β or γ-mercapto acids; its second end is another unit of α, β or γ-amino acid or α, β or γ-hydroxy acid or α, β or γ-mercapto acid, here abbreviated AAAn. During the course of the method, the peptide or protein or peptidomimetic chain lengthens by elongation or successive iteration, and during each of these steps another unit of activated α, β or γ-amino acid or α, β or γ-hydroxy acid or α, β or γ-mercapto acid is added, here abbreviated AAA(n+1), to said second end (primary or secondary amine, alcohol or free thiol). This reaction sequence is shown in reaction diagram no 2 below.

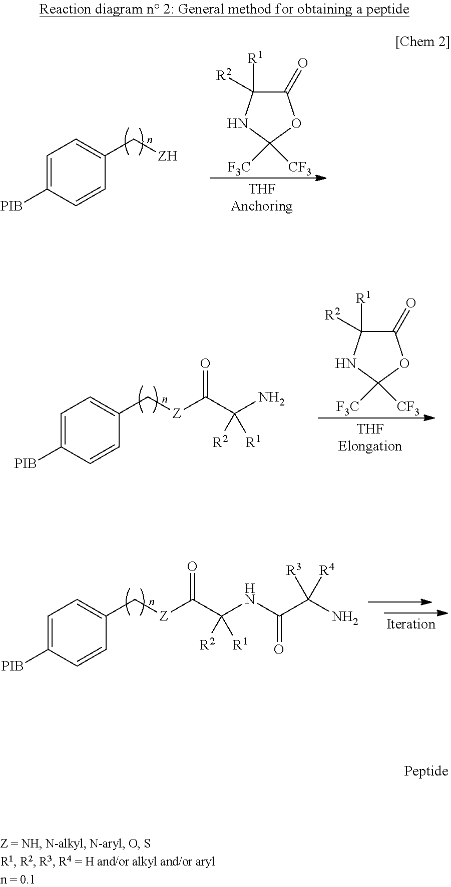

According to yet another feature of the invention, it is possible to use in said peptide or protein or peptidomimetic chain, natural and/or unnatural and/or synthetic α, β or γ-amino acids and/or α, β or γ-hydroxy acids and/or α, β or γ-mercapto acids.

According to yet another feature of the invention, it is possible to use in said peptidomimetic chain one or more units of Q^{a}-E-Q^{b} type molecules, having at least two functional groups, which are identical or different, and which are selected from electrophilic groups and/or nucleophilic groups, and which are separated by a spacer unit E. The groups Q^{a} and/or Q^{b} may or may not be terminal groups. The spacer E can be an entity selected from the group made up of:
aliphatic chains (branched or not and unsaturated or not);
aryls or heteroaryls (substituted or not).

Advantageously, the molecules of the Q^{a}-E-Q^{b} type carry a terminal function selected from the group made up of the primary amine function, the secondary amine function, the hydroxyl function or the thiol function.

It can easily be seen that said α, β or γ-amino acids, said α, β or γ-hydroxy acids and said α, β or γ-mercapto acids represent particular cases of bifunctional molecules of the Q^{a}-E-Q^{b} type. The same applies to said δ-amino acids, said δ-hydroxy acids and said δ-mercapto acids, which, however, are not necessarily involved in the reaction in their activated form, like the other bifunctional molecules which are not α, β or γ-amino acids, α, β or γ-hydroxy acids or α, β or γ-mercapto acids.

The bifunctional molecule Q^{a}-E-Q^{b} can have a molecular structure, selected in particular from epoxides, aziridines, thiiranes. Thus, according to the invention, peptidomimetics including an epoxy-succinate group, like peptide E-64, or azirido peptides, like Miraziridine, can be prepared.

Some examples are given here for bifunctional Q^{a}-E-Q^{b} type molecules which can be used within the context of the present invention: sarcosine, 2-(1-aminoethyl)-1,3-oxazole-4-carboxylic acid and (2R,3R,4R)-3-hydroxy-2,4,6-trimethyl-heptanoic acid.

Said bifunctional molecule Q^{a}-E-Q^{b} can in particular be an amino acid according to the definition which is given below. It can also be a peptide, for example a dipeptide, a tripeptide, a tetrapeptide, a pentapeptide, a hexapeptide, a heptapeptide, an octapeptide, a nonapeptide, a decapeptide, or an even longer peptide.

These bifunctional molecules can be introduced into the peptide or protein or peptidomimetic chain by known chemical reactions. They do not carry a protection group on a terminal function selected from the group made up of the primary amine function, the secondary amine function, the hydroxyl function or the thiol function. For example, if said bifunctional molecule is an amino acid or a peptide, it does not carry N-terminal protection; it is possible to protect its side chains or side functions, which are not modified during the elongation of the peptide.

As described above, said α, β or γ-amino acids, said α, β or γ-hydroxy acids and said α, β or γ-mercapto acids are preferably used in their activated forms.

The units derived from bifunctional molecules which are not selected from α, β or γ-amino acids, α, β or γ-hydroxy acids and α, β or γ-mercapto acids, can advantageously be attached to the C-terminal end of said peptidomimetic, or on the terminal end (in particular by functionalization of the primary or secondary amine, hydroxyl or thiol function), or on the side chain (of at least one α, β or γ-amino acid or α, β or γ-hydroxy acid or α, β or γ-mercapto acid), or between two units selected from α, β or γ-amino acids, α, β or γ-hydroxy acids and α, β or γ-mercapto acids.

According to an advantageous embodiment, the number of units resulting from bifunctional molecules which are not selected from α, β or γ-amino acids, α, β or γ-hydroxy acids and α, β or γ-mercapto acids does not exceed 50% in number, and preferably does not exceed 25% in number.

According to yet another feature of the invention, at least one step wherein said peptide or protein or peptidomimetic chain is attached to said anchoring molecule and is purified from the reaction medium by extraction in an organic solvent (such as cyclohexane, heptane(s) or any other suitable solvent) immiscible with water (or a water/ethanol mixture or a water/acetonitrile mixture) or by filtration on silica.

According to yet another feature of the invention, it allows to obtain peptides or proteins or peptidometics of high purity, after deprotection of the side chains (if necessary), then detachment of their anchoring molecule after the last iteration step, to be used according to their destination, for example as an active ingredient for preclinical trials, clinical care or any other applications.

According to yet another feature of the invention, the anchoring molecules can be reused (recycled) in the method according to the invention.

A second object of the present invention is a molecule capable of being obtained by the method according to the invention. Said molecule comprises an α, β or γ-amino acid or α, β or γ-hydroxy acid or α, β or γ-mercapto acid or peptide or protein or peptidomimetic attached to an anchoring molecule.

### DESCRIPTION

### 1. Definitions

In the context of the present invention, “amino acid” means: natural amino acids and unnatural or synthetic amino acids. “Natural” amino acids comprise the L form of proteinogenic amino acids called standard proteinogenic amino acids that can be found in proteins of natural origin, that is to say: alanine (Ala), arginine (Arg), asparagine (Asn), aspartic acid (Asp), cysteine (Cys), glutamine (Gln), glutamic acid (Glu), glycine (Gly), histidine (His), isoleucine (Ile), leucine (Leu), lysine (Lys), methionine (Met), phenylalanine (Phe), proline (Pro), serine (Ser), threonine (Thr), tryptophan (Trp), tyrosine (Tyr) and valine (Val). They also comprise other proteinogenic amino acids, and in particular pyrrolysine and selenocysteine.

“Unnatural” amino acids comprise the D-form of the natural amino acids defined above, the homo forms of certain natural amino acids (such as: arginine, lysine, phenylalanine and serine), and the nor forms of leucine and valine.

“Unnatural” amino acids also comprise all synthetic amino acids. They also comprise unnatural amino acids, such as:

Abu=2-aminobutyric acid CH₃—CH₂—CH(COOH)(NH₂);

iPr=isopropyl-lysine (CH₃)₂C—NH—(CH₂)₄—CH(COOH)(NH₂);

Aib=2-aminoisobutyric acid;

F-trp=N-formyl-tryptophan;

Orn=ornithine;

Nal(2′)=2-naphthylalanine.

This list is obviously not exhaustive.

It is also possible to use natural or unnatural unsaturated α and β amino acids.

In the context of the present invention, the term “activated amino acid” is used here to designate activated α, β or γ-amino acids respectively in the form of 2,2-bis(trifluoromethyl)-1,3-oxazolidin-5-one or (2,2-bis(trifluoromethyl)-1,3-oxazinan-6-one or 2,2-bis(trifluoromethyl)-1,3-oxazepan-7-one, and derivatives thereof, with the possibility or not of a protection group on the side chain.

The present invention is also applicable to the synthesis of peptidomimetics. The precursors used for this synthesis are defined as follows:

The term “α, β or γ-hydroxy acid” is used here according to the terminology rules of IUPAC, known to the person skilled in the art. Examples are compounds such as lactic acid, malic acid, tartaric acid, salicylic acid or γ-hydroxybutyric acid, which are found in nature. In the context of the present invention, it is also possible to use all the “unnatural” α, β or γ-hydroxy acids which also comprise all the synthetic α, β or γ-hydroxy acids.

The term “activated α, β or γ-hydroxy acid” designates all natural and/or unnatural and/or synthetic α, β or γ-hydroxy acids, which were activated respectively in the form of 2,2-bis(trifluoromethyl)-1,3-dioxolan-4-one or 2,2-bis(trifluoromethyl)-1,3-dioxan-4-one or 2,2-bis(trifluoromethyl)-1,3-dioxepan-4-one, and derivatives thereof, with the possibility or not of a protection group on the side chain.

The term “α, β or γ-mercapto acid” is used here according to the terminology rules of IUPAC, known to the person skilled in the art. Examples are compounds such as thioglycolic acid, 3-mercaptopropionic acid, mercaptobutanoic acid. In the context of the present invention, it is also possible to use all the “unnatural” α, β or γ-mercapto acids which also comprise all the synthetic α, β or γ-mercapto acids.

The term “activated α, β or γ-mercapto acid” designates all compounds resulting from the activation of (natural, unnatural or synthetic) α, β or γ-mercapto acids in the form of 2,2-bis(trifluoromethyl)-1,3-oxathiolan-5-one or 2,2-bis(trifluoromethyl)-1,3-oxathian-6-one or 2,2-bis(trifluoromethyl)-1,3-oxathiepan-7-one, and derivatives thereof, with the possibility or not of a protection group on the side chain.

The person skilled in the art knows that in this context, the designation α, β, γ and δ refers to the position of the carbon substituted by the (primary or secondary) amine or hydroxyl or thiol function with respect to the carbon of the carboxylic acid function (C-terminal).

The term “peptidomimetic” is used according to the prior art as a functional term for a molecule capable of mimicking or blocking a peptide with respect to its interaction with a specific receptor. In particular, a peptidomimetic may comprise units which are not amino acids.

The abbreviations “DMF”, “DMSO” and “THF”, well known to chemists, designate, respectively, dimethylformamide, dimethylsulfoxide and tetrahydrofuran.

### 2. Detailed Description

A first essential feature of the method according to the invention is the use of activated α, β or γ-amino acids or α, β or γ-hydroxy acids or α, β or γ-mercapto acids respectively in the form of 2,2-bis(trifluoromethyl)-1,3-oxazolidin-5-one or (2,2-bis(trifluoromethyl)-1,3-oxazinan-6-one or 2,2-bis(trifluoromethyl)-1,3-oxazepan-7-one or 2,2-bis(trifluoromethyl)-1,3-dioxolan-4-one or 2,2-bis(trifluoromethyl)-1,3-dioxan-4-one or 2,2-bis(trifluoromethyl)-1,3-dioxepan-4-one or 2,2-bis(trifluoromethyl)-1,3-oxathiolan-5-one or 2,2-bis(trifluoromethyl)-1,3-oxathian-6-one or 2,2-bis(trifluoromethyl)-1,3-oxathiepan-7-one and derivatives thereof, in the presence of an anchoring molecule soluble in an organic solvent. “Organic solvent” here means any inert liquid solvent (or mixture) capable of (hot and/or cold) dissolving the reactants. Applicable solvents comprise, without limitation, halogenated or non-halogenated hydrocarbons.

Activated α, β or γ-amino acids or α, β or γ-hydroxy acids or α, β or γ-mercapto acids respectively in the form of 2,2-bis(trifluoromethyl)-1,3-oxazolidin-5-one or (2,2-bis(trifluoromethyl)-1,3-oxazinan-6-one or 2,2-bis(trifluoromethyl)-1,3-oxazepan-7-one or 2,2-bis(trifluoromethyl)-1,3-dioxolan-4-one or 2,2-bis(trifluoromethyl)-1,3-dioxan-4-one or 2,2-bis(trifluoromethyl)-1,3-dioxepan-4-one or 2,2-bis(trifluoromethyl)-1,3-oxathiolan-5-one or 2,2-bis(trifluoromethyl)-1,3-oxathian-6-one or 2,2-bis(trifluoromethyl)-1,3-oxazepan-7-one and derivatives thereof, are prepared according to known methods, from α, β or γ-amino acids or α, β or γ-hydroxy acids or α, β or γ-mercapto acids, which are natural or unnatural (having a side chain (protected or not)) and hexafluoroacetone. Reaction diagram no 3 represents the activation of different acids, it is a reaction known as such:

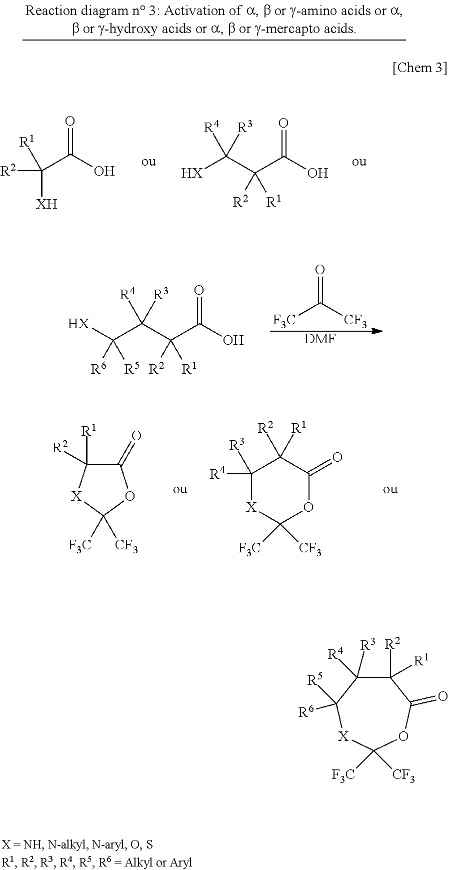

According to one feature of the invention, which will be described in greater detail below, the anchoring molecules (or protection groups or solubilizing molecules) are polyolefins or more specifically polyolefin oligomers (polyolefins also being called polyalkenes) and derivatives thereof, that is to say they are functionalized.

According to another feature of the invention, this method for synthesizing peptides or proteins or peptidomimetics (protected or not on their side chains), in the liquid phase, is characterized in that use is made of an anchoring molecule and an activated α, β or γ-amino acid or α, β or γ-hydroxy acid or α, β or γ-mercapto acid respectively in the form of 2,2-bis(trifluoromethyl)-1,3-oxazolidin-5-one or (2,2-bis(trifluoromethyl)-1,3-oxazinan-6-one or 2,2-bis(trifluoromethyl)-1,3-oxazepan-7-one or 2,2-bis(trifluoromethyl)-1,3-dioxolan-4-one or 2,2-bis(trifluoromethyl)-1,3-dioxan-4-one or 2,2-bis(trifluoromethyl)-1,3-dioxepan-4-one or 2,2-bis(trifluoromethyl)-1,3-oxathiolan-5-one or 2,2-bis(trifluoromethyl)-1,3-oxathian-6-one or 2,2-bis(trifluoromethyl)-1,3-oxathiepan-7-one or derivatives thereof. A covalent bond is then formed between these two molecular species. The elongation/iteration step consists in adding or condensing the following activated α, β or γ-amino acids or α, β or γ-hydroxy acids or α, β or γ-mercapto acids, which are optionally protected on their side chain (in the form of ester, ether, thioester, thioether or any other chemical functions compatible with the present method). Thus, the anchoring molecule acts as a protection group for the carboxylic acid function (C-terminal) of the first α, β or γ-amino acid or α, β or γ-hydroxy acid or α, β or γ-mercapto acid.

According to another feature of the invention, this method for synthesizing peptide or protein or peptidomimetic can be carried out using a fragment of a peptide or protein or peptidomimetic suitably protected and an α, β or γ-amino acid or α, β or γ-hydroxy acid or α, β or γ-mercapto acid or peptide or protein or peptidomimetic, anchored on a PIB molecule, allowing, after coupling, to obtain a longer peptide or protein or peptidomimetic.

According to another feature of the invention, this method for synthesizing peptide or protein or peptidomimetic can be carried out using molecules Q^{a}-E-Q^{b} having at least two functional groups Q^{a} and Q^{b}, which are identical or different, and which are selected from the electrophilic and/or nucleophilic chemical functions. Examples of these structures are styrene oxide, aminothiophenoles or 1-azido-4-(bromomethyl) benzene. These molecules can be directly attached to the anchoring molecule or introduced during synthesis on the (primary or secondary) amine function or hydroxyl or thiol, α, β or γ-amino acids or α, β or γ-hydroxy acids or α, β or γ-mercapto acids or anchored peptides or proteins or peptidomimetics.

The method according to the invention can be carried out in any inert liquid solvent (or mixture) capable of dissolving the (halogenated or non-halogenated) reactants, at a temperature typically comprised between about −20° C. and about 150° C., in a reactor (in batch or in flow).

According to another feature of the invention the α, β or γ-amino acid or α, β or γ-hydroxy acid or α, β or γ-mercapto acid or peptide or protein or peptidomimetic anchored on a PIB molecule is characterized in that the terminal function of said α, β or γ-amino acid or α, β or γ-hydroxy acid or α, β or γ-mercapto acid or peptide or protein or peptidomimetic or any other molecule having at least two functional groups is bonded by a covalent bond (ester, ether, amide, thioester or any other chemical functions), thus giving a very low solubility in water (<30 mg/ml). It is in this sense that the PIB derivative acts as a liquid carrier or a solubilizing molecule for the synthesis of peptides or proteins or peptidomimetics.

By way of illustration, reaction diagram no 4 shows the reaction of an activated amino acid in the form of 2,2-bis(trifluoromethyl)-1,3-oxazolidin-5-one with a polyisobutene derivative (abbreviated PIB) which is terminated by a phenol function. In this case, the α-amino acid is L-phenylalanine (Phe).

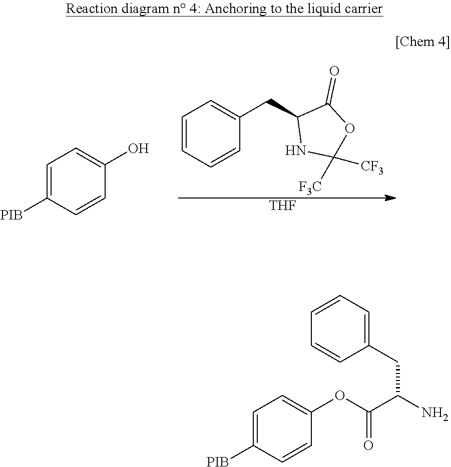

Thus, the first α-amino acid of the future peptide is attached to the anchoring molecule via an ester-type covalent bond.

Reaction diagram 5 shows the elongation or iteration step, that is to say the attachment of a second amino acid unit, to the first amino acid attached to the anchoring molecule. In this case, that second α-amino acid is L-tryptophan (Trp).

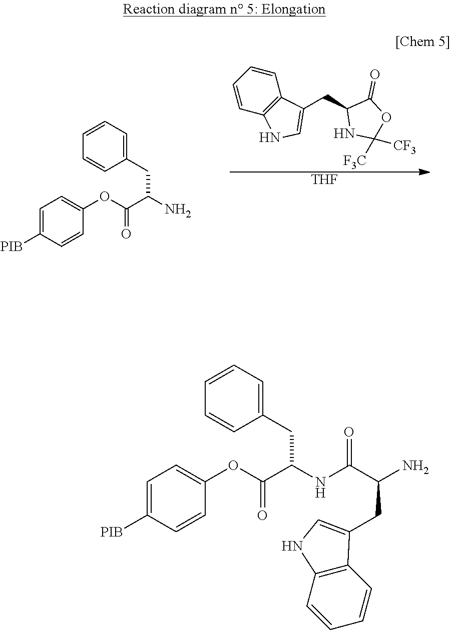

It can be easily seen that this method allows, by successive iterations, to add units of α, β or γ-amino acids or α, β or γ-hydroxy acids or α, β or γ-mercapto acids on the last α, β or γ-amino acid or α, β or γ-hydroxy acid or α, β or γ-mercapto acid or peptide or protein or peptidomimetic attached to the PIB derivative, during synthesis, to obtain a peptide or protein or peptidomimetic having the desired sequence. The peptide or protein or peptidomimetic being chemically bonded to the anchoring molecule, it can be separated at any time, and in particular after the last iteration step, from all polar products by extraction in an organic solvent such as hexane(s) or cyclohexane and water or in a water/ethanol or water/acetonitrile mixture. At the end of this iteration sequence, and optionally after deprotection of the side chains, the peptide or protein or peptidomimetic can be detached from the anchoring molecule; thus the peptide or protein or peptidomimetic loses its solubility in an apolar solvent, and can be separated from the anchoring molecule, for use in accordance with its intended purpose.

According to another feature of the invention, the derivation (or anchoring) of an α, β or γ-amino acid or α, β or γ-hydroxy acid or α, β or γ-mercapto acid or peptide or protein or peptidomimetic (protected or not on its side chain(s)) with a PIB derivative indeed leads to a significant increase in the solubility of said α, β or γ-amino acid or α, β acid or γ-hydroxy acid or α, β or γ-mercapto acid or peptide or protein or peptidomimetic anchored in organic liquid phase. More specifically, these α, β or γ-amino acids or α, β or γ-hydroxy acids or α, β or γ-mercapto acids or peptides or proteins or peptidomimetics anchored on a PIB derivative become soluble in organic solvents, such as halogenated solvents (methylene chloride, chloroform), ethyl acetate, tetrahydrofuran, 2-methyletetrahydrofuran, isooctane, cyclohexane, hexane (s), methylcyclohexane, methyl tert-butyl ether propylene carbonate or aromatic solvents such as benzene or toluene or any other suitable solvent. Thus, α, β or γ-amino acids or α, β or γ-hydroxy acids or α, β or γ-mercapto acids or peptides or proteins or peptidomimetics anchored on a PIB derivative have a high partition coefficient for the organic phase during extraction/decantation, thus allowing simple and rapid purification. At the same time, their solubility in solvents such as water or a water/ethanol or water/acetonitrile mixture is very low.

According to another feature of the invention, the reaction between a PIB derivative and the first activated α, β or γ-amino acid or α, β or γ-hydroxy acid or α, β or γ-mercapto acid, optionally having a side chain protected or not (ester, amide, thioester or any other chemical functions), leads to a product whose solubility in water is low (<30 mg/ml).

Thus, when an α, β or γ-amino acid or α, β or γ-hydroxy acid or α, β or γ-mercapto acid or peptide or protein or peptidomimetic is attached to a PIB derivative, the latter acts as a liquid carrier (or anchoring molecule or solubilizing molecule) because the product of this reaction becomes soluble in organic solvents but remains insoluble in solvents such as water or a water/ethanol or water/acetonitrile mixture; this allows its separation from the reaction mixture by phase separation.

Reaction diagram no 6 shows an example of the step of detaching an octapeptide having the sequence Phe-Tpr-Cys(Bzl)-Trp-Cys(Bzl)-Trp-Trp-Cys(Bzl)-NH₂, from the anchoring molecule (PIB derivative terminated by a phenol function), on which the peptide is anchored by the carboxylic acid function (C-terminal) of the L-phenylalanine. The side chains of L-cysteine residues are protected by a benzyl group (Bzl). The free peptide is insoluble in apolar solvents (that is to say cyclohexane, hexane(s)), which allows it to be easily separated from the anchoring molecule. The anchoring molecule can be recovered for reuse in the method.

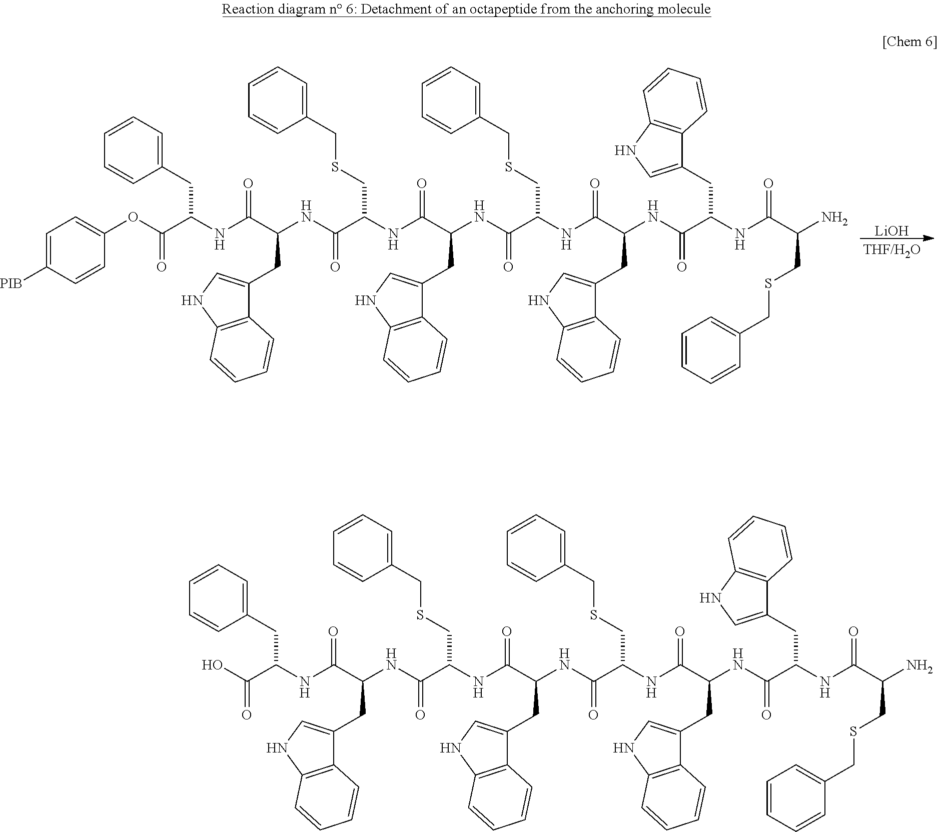

The peptide precipitates in solvents such as: ethyl ether, cyclohexane or any other suitable solvent. It can then be used in accordance with its intended purpose.

A second essential feature of the method for preparing peptides or proteins or peptidomimetics according to the invention will now be described, namely the use of anchoring molecules soluble in some organic solvents such as: ethyl acetate, tetrahydrofuran, 2-methyletetrahydrofuran, isooctane, cyclohexane, hexane (s), methylcyclohexane, methyl tert-butyl ether or halogenated solvents.

Advantageously, the method according to the invention uses polyolefins or more specifically polyolefin oligomers (polyolefins also being called polyalkenes), and derivatives thereof as anchoring molecules or liquid carrier or protection group, whether for the carboxylic acid function (C-terminal) of α, β or γ-amino acid or α, β or γ-hydroxy acid or α, β or γ-mercapto acid or peptide or protein or peptidomimetic, or of the side chain of said α, β acid or γ-amino or α, β or γ-hydroxy acid or α, β or γ-mercapto acid or peptide or protein or peptidomimetic (in the form of ester, amide, ether, thioester, thioether or any other suitable chemical functions) in liquid phase. Polyolefin molecules comprise a chain of carbon atoms bonded together by single bonds. They may comprise branches made up of identical or different alkyl groups, but preferably identical alkyl groups. Preferably, the polymers consist of a number of monomer units of at least 10 and preferably comprised between 15 and 350. Homopolymers are preferred, but (saturated or unsaturated) copolymers can be used. In the case of unsaturated polymers or copolymers, the number of unsaturated bonds in the chain of carbon atoms advantageously does not exceed 5%, and preferably does not exceed 3%.

Preferably, they are derivatives of polyisobutenes (PIB), a class of polymers known since the 1930s of the last century, but it is also possible to use derivatives of polypropylenes.

These anchoring molecules are preferably used in the method according to the invention in the form of functionalized derivatives, as will be explained in greater detail below. Diagram no 7 shows a number of PIB derivatives with their functionalizations which are suitable as a liquid carrier for carrying out the present invention.

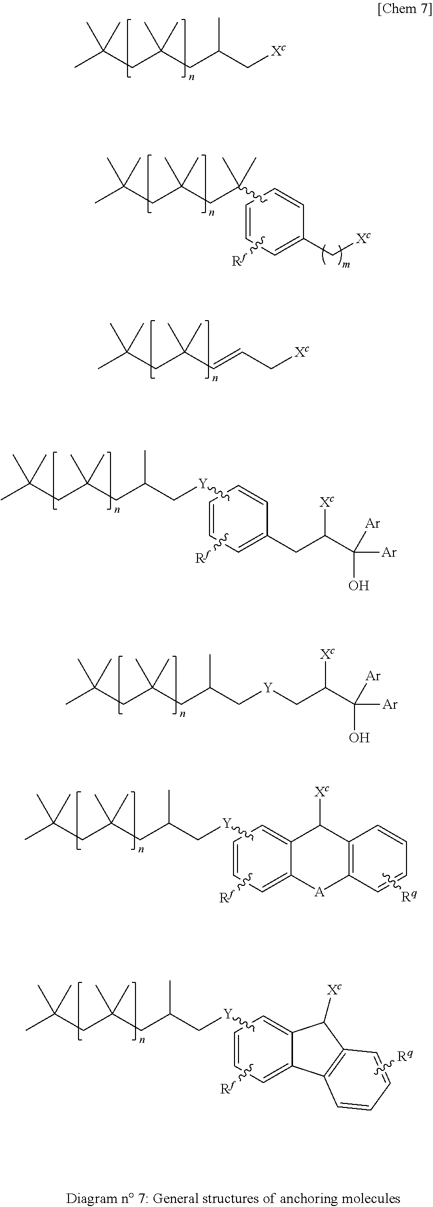

In these formulas:
X^{c} is a group selected from the group made up of —OH, —NH₂, —NHR^{a} (R^{a}=alkyl or aryl), —SH;
Ar is an aromatic or heteroaromatic group, substituted or not;
A is either absent or a group selected from the group made up of: CH₂, CH₂—CH₂, S;
R^{f} is a group selected from the group made up of H, aryl, hetero-aryl, alkyl, O-alkyl, O-aryl, O-hetero-aryl;
R^{q} is a group selected from the group made up of H, alkyl, O-alkyl, aryl, hetero-aryl, O-aryl, O-hetero-aryl;
the number n is an integer which is typically greater than 10, and advantageously comprised between 15 and 350.

The number m is either 0 or 1. In particular, the group X^{c} can be a function in a primary or secondary amine, an alcohol, a thiol or a phenol.

According to the invention, these anchoring molecules are bonded to the carboxylic acid function of an α, β or γ-amino acid or α, β or γ-hydroxy acid or α, β or γ-mercapto acid (C-terminal) or all chemical functions of a molecule having at least two functional groups via a covalent bond. This assumes that the anchoring molecules are engaged in a suitably functionalized form, which is referred to in the present description as “PIB derivatives”. This term also encompasses derivatives of anchoring molecules which are not derivatives of polyisobutene, but which are derivatives of other polyolefins as defined above; it encompasses in particular derivatives of polyolefin oligomers. This functionalization of the anchoring molecule is generally a terminal functionalization, preferably at one of the ends of the chain of carbon atoms; it is described below.

According to the invention, the α, β or γ-amino acids or α, β or γ-hydroxy acids or α, β or γ-mercapto acids or peptides or proteins or peptidomimetics can be functionalized on their side chains with PIB derivatives, in the form of ester, ether, thioether, thioester or any other chemical functions compatible with the present method. This amounts to giving the PIB derivatives a role of protection group(s) of the side chain(s) of said α, β or γ-amino acids or α, β or γ-hydroxy acids or α, β or γ-mercapto acids or peptides or proteins or peptidomimetics.

The chains of polyolefins or polyolefin oligomers or polyalkenes used as anchoring molecules are typically characterized by a weight average molecular weight, but it is also possible to use chains of polyolefins or polyolefin oligomers or polyalkenes known as “homogeneous” chains which include identical molecules of a given chain length.

More specifically, the method for synthesizing peptides or proteins or peptidomimetics, optionally protected on their side chains, in the liquid phase (solution) according to the invention, is characterized by the fact that an α, β or γ-amino acid or α, β or γ-hydroxy acid or α, β or γ-mercapto acid or peptide or protein or peptidomimetic is dissolved in an organic medium by a PIB derivative bonded to the carboxylic acid function of the α, β or γ-amino acid or α, β or γ-hydroxy acid or α, β or γ-mercapto acid or peptide or protein or peptidomimetic or any other molecule having at least two functional groups. The PIB derivative acts as an anchoring molecule (here also called “liquid carrier” or “solubilizing molecule”) of α, β or γ-amino acid or α, β or γ-hydroxy acid or α, β or γ-mercapto acid or peptide or protein or peptidomimetic or any other molecule having at least two functional groups. Said peptide or protein or peptidomimetic attached to this anchoring molecule is synthesized by successive attachment of α, β or γ-amino acids or α, β or γ-hydroxy acids or α, β or γ-mercapto acids or any other molecules having at least two functional groups on the last α, β or γ-amino acid or α, β or γ-hydroxy acid or α, β or γ-mercapto acid or any other molecule having at least two functional groups. Thus, the anchoring molecule also serves as a protection group for the carboxylic acid function (C-terminal) during the synthesis of the peptide or protein or peptidomimetic in successive iterations.

The carboxylic acid function (C-terminal) of said α, β or γ-amino acid or α, β or γ-hydroxy acid or α, β or γ-mercapto acid or peptide or protein or peptidomimetic, optionally protected on its lateral chain(s), is bonded by a covalent bond of ester, amide, thioester, or any other covalent chemical bond, to a lipophilic PIB derivative, giving a very low solubility in water (<30 mg/ml). It is in this sense that the PIB derivative acts as a liquid carrier or a solubilizing molecule for the synthesis of peptides or proteins or peptidomimetics.

This derivative of α, β or γ-amino acid or α, β or γ-hydroxy acid or α, β or γ-mercapto acid or peptide or protein or peptidomimetic (protected or not on its side chains) with a PIB derivative significantly increases the solubility of said α, β or γ-amino acid or α, β or γ-hydroxy acid or α, β or γ-mercapto acid or peptide or protein or anchored peptidomimetic, in organic liquid phase. More specifically, these α, β or γ-amino acids or α, β or γ-hydroxy acids or α, β or γ-mercapto acids or peptides or proteins or peptidomimetics anchored on the PIB derivative become soluble in organic solvents such as: halogenated solvents (methylene chloride, chloroform), ethyl acetate, tetrahydrofuran, 2-methyletetrahydrofuran, isooctane, cyclohexane, hexane(s), methylcyclohexane, methyl tert-butyl ether or aromatic solvents such as benzene or toluene or any other suitable solvent. As a result, α, β or γ-amino acids or α, β or γ-hydroxy acids or α, β or γ-mercapto acids or peptides or proteins or peptidomimetics attached to a PIB derivative have a high partition coefficient for the organic phase during extraction/decantation in the presence of cyclohexane or hexane(s) and water or a water/ethanol or else water/acetonitrile mixture, thus allowing their simple and rapid purification.

In one embodiment of the method for synthesizing peptides or proteins or peptidomimetics (protected or not on their side chains), in the liquid phase according to the invention, the starting point is a molecule having at least two functional groups or an activated α, β or γ-amino acid or α, β or γ-hydroxy acid or α, β or γ-mercapto acid respectively in the form of 2,2-bis(trifluoromethyl)-1,3-oxazolidin-5-one or (2,2-bis(trifluoromethyl)-1,3-oxazinan-6-one or 2,2-bis(trifluoromethyl)-1,3-oxazepan-7-one or 2,2-bis(trifluoromethyl)-1,3-dioxolan-4-one or 2,2-bis(trifluoromethyl)-1,3-dioxan-4-one or 2,2-bis(trifluoromethyl)-1,3-dioxepan-4-one or 2,2-bis(trifluoromethyl)-1,3-oxathiolan-5-one or 2,2-bis(trifluoromethyl)-1,3-oxathian-6-one or 2,2-bis(trifluoromethyl)-1,3-oxathiepan-7-one or derivatives thereof, which will be bonded to one of the anchoring molecules as defined above, via a covalent bond, and which is added or condensed, by successive iterations, molecules having at least two functional groups or α, β or γ-amino acids or α, β or γ-hydroxy acids or α, β or γ-mercapto acids which are activated and optionally protected on their side chains.

The method for synthesizing peptide or protein or peptidomimetic according to the invention can be carried out using a fragment of a peptide or protein or peptidomimetic suitably protected on its side chains and a peptide or protein or peptidomimetic sequence anchored on a PIB molecule allowing, after coupling, to obtain a longer peptide or protein or peptidomimetic.

The method for synthesizing peptide or protein or peptidomimetic according to the invention can be carried out using molecules having at least two functional groups, namely a group Q^{a} and a group Q^{b}, which may be identical or different, and which are selected from electrophilic groups and/or nucleophilic groups. By way of example, in a first embodiment Q^{a} can be an electrophilic group, and Q^{b} can be a nucleophilic group, or alternatively, in a second embodiment, Q^{a} can be a first electrophilic group and Q^{b} a second electrophilic group, or alternatively, in a third embodiment, Q^{a} can be a first nucleophilic group and Q^{b} a second nucleophilic group; in variants, Q^{a} and Q^{b} can also designate the same electrophilic group, or else the same nucleophilic group. These molecules having at least two functional groups can be directly anchored on the anchoring molecule or introduced during synthesis on the (primary or secondary) amine function or hydroxyl or thiol, α, β or γ-amino acids or α, β or γ-hydroxy acids or α, β or γ-mercapto acids or peptides or proteins or anchored peptidomimetics.

Advantageously, a slight stoichiometric excess of the activated α, β or γ-amino acid or α, β or γ-hydroxy acid or α, β or γ-mercapto acid is used during each anchoring, elongation and/or iteration step.

These bifunctional molecules can be introduced into the peptide or protein or peptidomimetic chain by known chemical reactions. If necessary, they can be protected or masked (on its nucleophilic function or any other chemical functions requiring it, by means of known reactions) and activated by known techniques.

The units derived from bifunctional molecules which are not selected from α, β or γ-amino acids or α, β or γ-hydroxy acids or α, β or γ-mercapto acids can advantageously be attached to the C-terminal end of said peptide or protein or peptidomimetic, or on the N, O or S-terminal end (in particular by functionalization of the amine, hydroxyl or thiol function), or else on the side chain, or alternatively between two units selected from α, β or γ-amino acids or α, β or γ-hydroxy acids or α, β or γ-mercapto acids.

According to an advantageous embodiment, the number of units derived from bifunctional molecules which are not selected from α, β or γ-amino acids or α, β or γ-hydroxy acids or α, β or γ-mercapto acids does not exceed 50% in number of units, and preferably does not exceed 25% in number of units, and even more preferably does not exceed 10% in number of units.

For example, semaglutide, a peptidomimetic including on a side chain an α-aminobutyric acid unit, can be prepared using the method according to the invention.

A list of molecules of α, β, γ or 5-amino acid type is given here which can be used as units in the context of the method according to the invention, in addition to the amino acids already mentioned above: 5-amino levulinic acid, γ-aminobutyric acid, β-aminobutyric acid (also known by the acronym BABA), β-alanine, β-lysine, β-aminoisobutyric acid, β-N-Methylamino-L-alanine, (2S,3S,8S,9S)-3-amino-9-methoxy-2,6,8-trimethyl-10-phenyldeca-4,6-dienoic acid (also known as ADDA), (2R)-2-(methylamino)butanedioic acid (also known as NMDA) and 4-amino-3-hydroxybutanoic acid.

A list of molecules of α, β, γ or 5-hydroxy acid type is given here which can be used as units in the context of the method according to the invention: 4-hydroxybutanoic acid, 2-(hydroxymethyl)-3-methylbutanoic acid and (2R,3R,4R)-3-hydroxy-2,4,6-trimethylheptanoic acid.

A list of molecules of α, β, γ or 5-mercapto acid type is given here which can be used as units within the context of the method according to the invention: 4-sulfanylbutanoic acid, 2-cyclopropyl-3-sulfanylpropanoic acid, 2-cyclobutyl-3-sulfanylpropanoic acid and 2-(2-sulfanylphenyl)butanoic acid.

The method according to the invention has many advantages.

A first advantage is that it allows the production of peptides or proteins or peptidomimetics (protected or not on their side chains) bonded to the anchoring molecule in the organic liquid phase.

A second advantage is that it allows to obtain anchored peptides or proteins or peptidomimetics (protected or not on their side chains) of high purity by a simple washing (extraction) in an apolar organic solvent and with water or in a water/ethanol or else water/acetonitrile mixture or by filtration, thus causing the elimination of by-products (salts, acids or any other molecular species) which are not bonded to the derivative of polyolefins or polyolefin oligomers or polyalkenes such as excess reagents. Organic solvents such as cyclohexane, heptane, hexane(s) which have flash points<15° C., are suitable for solubilizing the derivatives of polyolefins or polyolefin oligomers or polyalkenes during extraction or washing. The method according to the invention therefore allows to limit the purification steps which are necessary in the methods of the prior art.

A third advantage, which is particularly important, is that the method according to the invention allows to synthesize peptides or proteins or peptidomimetics, by adjusting the length of the derivative of polyolefins or polyolefin oligomers or polyalkenes, that is to say by making them more lipophilic.

A fourth advantage is the possibility of controlling the purity of the peptide or protein or peptidomimetic during synthesis, at any time, by taking an aliquot followed by analysis by the various techniques known to the person skilled in the art (such as mass spectrometry, high performance liquid chromatography, proton or carbon-13 nuclear magnetic resonance).

A fifth advantage lies in the fact that it is not necessary to use a protection group for the (primary or secondary) amine function or hydroxyl or thiol, respectively, α, β or γ-amino acids or α, β or γ-hydroxy acids or α, β or γ-mercapto acids which, generally, costs two steps (protection and deprotection). More generally, the method according to the invention allows an optimal economy of atoms because it does not involve either protection groups for the (primary or secondary) amine or hydroxyl or thiol function of the corresponding acids, or coupling agents. This economy of atoms and steps of the method according to the invention generates, in industrial reality, financial savings while reducing the generation of waste, which is a favorable environmental factor unlike current methods.

A sixth advantage of the invention lies in the fact that the activation of the carboxylic acid function (C-terminal) is concomitant with the protection of the (primary or secondary) amine or hydroxyl or thiol function, therefore reducing the number of steps.

A seventh particularly interesting advantage of the invention lies in obtaining peptides or proteins or peptidomimetics of high purity after the cleavage of the protection groups of the side chains, then of the anchoring molecule. This avoids purifying the synthesized peptide or protein or peptidomimetic. As a result, additional savings are generated over known methods. This further limits the environmental impact of the production of peptides or proteins or peptidomimetics.

An eighth advantage of the invention lies in the possibility of accessing peptides or proteins or peptidomimetics of larger sizes, either by modulating the size of the liquid carrier or by introducing it on one or more side chains of activated α, β or γ-amino acids or α, β or γ-hydroxy acids or α, β or γ-mercapto acids.

Other advantages are the possibility of automating the method according to the invention and the possibility of recycling the extraction solvents and the anchoring molecules (polyolefins or polyolefin oligomers or polyalkenes). Indeed, when the series of iterations to obtain the sequence of the target peptide or protein or peptidomimetic is completed, the latter is deprotected from its protection groups of the side chains and finally, of the anchoring molecule by one of the reactions usually used in peptide synthesis, such as hydrolysis, saponification, hydrogenolysis or any other reaction compatible with the present method.

Thanks to their high purity, the peptides or proteins or peptidomimetics produced by this method can be used as pharmaceutical products (drugs and vaccines), cosmetics, phytosanitary products, food products or as intermediates to synthesize such products.

### Example

An octapeptide was prepared using the method according to the invention.

In a first step the following amino acids were activated: activated L-phenylalanine (designated here as AAA1), activated L-tryptophan (designated here as AAA2) and activated L-cysteine (protected by a benzyl (Bzl) protection group) (designated here as AAA3). This reaction corresponds to reaction diagram no 3 above.

To a solution of the amino acid (10 mmol) in N—N-dimethylformamide (5 mL), at room temperature, equipped with a dry ice gas condenser and a bubbler, hexafluoroacetone was condensed in excess (>2 equivalents). After sixteen hours of stirring at room temperature, the reaction mixture was concentrated to dryness, and the residue was lyophilized. The crude product obtained was dissolved in dichloromethane, filtered then the solvent was removed under reduced pressure and lyophilized (three times). The 2,2-bis(trifluoromethyl)-1,3-oxazolidin-5-ones or activated amino acids were obtained in the form of oil or solid with yields comprised between 80-95%. Their formulas are given below in diagram no 8.

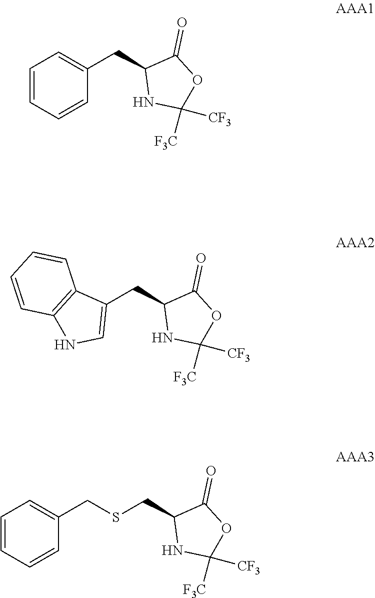 [SMILES: O=C1OC(C(F)(F)F)(C(F)(F)F)N[C@H]1Cc1ccccc1 ; O=C1OC(C(F)(F)F)(C(F)(F)F)N[C@H]1Cc1c[nH]c2ccccc12 ; O=C1OC(C(F)(F)F)(C(F)(F)F)N[C@H]1CSCc1ccccc1]

In a second step, an activated amino acid (activated L-phenylalanine referred to here as AAA1) is coupled to the anchoring molecule, in this case a PIB derivative. This reaction corresponds to reaction diagram no 4 above.

A solution of the PIB derivative (31.1 mg, 0.028 mmol) and the activated amino acid (here AAA1) (9.8 mg, 0.031 mmol) in a tetrahydrofuran/hexafluoroisopropanol mixture (2 mL) was heated to 50° C. for 2 hours then cooled to room temperature. Saturated aqueous sodium hydrogencarbonate solution (2 mL) was added to the reaction medium and the mixture was stirred at room temperature for thirty minutes. The reaction medium was extracted three times with cyclohexane, washed with brine, dried over sodium sulfate, filtered and concentrated under reduced pressure to lead to the H₂N-Phe-PIB derivative.

In a third step, the peptide is elongated by attaching another activated amino acid (here AAA2). This reaction corresponds to reaction diagram no 5 above.

A solution of the H₂N-Phe-PIB derivative (1 equivalent) and the following activated amino acid (AAA2) (1.1 equivalent) in tetrahydrofuran/hexafluoroisopropanol (2 mL, 9/1) was heated to 50° C. for 2 hours then cooled to room temperature. The reaction mixture was treated as before, to lead to the corresponding anchored dipeptide (H₂N-Trp-Phe-PIB), and so on. In this case, the iteration was repeated with the activated amino acid AAA3 under the same conditions to obtain the corresponding anchored tripeptide (H₂N-Cys (Bzl)-Trp-Phe-PIB).

Further iterations were then carried out until an anchored octapeptide of the sequence H₂N-Cys(Bzl)-Trp-Trp-Cys(Bzl)-Trp-Cys(Bzl)-Trp-Phe-PIB was obtained.

In a final step, the peptide is detached from the anchoring molecule. This reaction corresponds to reaction diagram no 6 above.

A solution of lithium hydroxide (1 M, 2 mL) was added to a solution of the anchored octapeptide (H₂N-Cys(Bzl)-Trp-Trp-Cys(Bzl)-Trp-Cys(Bzl)-Trp-Phe-PIB) (8 mg) in a tetrahydrofuran/water mixture (8:2) (2 mL), at room temperature. The reaction mixture was stirred at room temperature for 16 hours. The reaction medium was diluted with a dioxane/HCl solution and the precipitate was washed with cyclohexane.

What is claimed is:

1. A method for synthesizing peptides or proteins or peptidomimetics by successive elongation of the second end, which has a primary or secondary amine function, a hydroxyl function or a thiol function, of a peptide or protein or peptidomimetic chain by units, characterized in that:
said units are selected from the group made up of Q^{a}-E-Q^{b} type molecules, where Q^{a} and Q^{b} may be the same or different, and are selected from the electrophilic groups and the nucleophilic groups, and E represents a spacer;
the first end of said peptide or protein or peptidomimetic is attached by a covalent bond to an anchoring molecule soluble in organic solvents such as halogenated solvents (preferably methylene chloride or chloroform), ethyl acetate, tetrahydrofuran, 2-methyletetrahydrofuran, isooctane, cyclohexane, hexane(s), methylcyclohexane, methyl tert-butyl ether or aromatic solvents such as benzene or toluene;
said method does not involve protection groups for the primary amine or secondary amine or hydroxyl or thiol function.

2. The method according to claim 1, characterized in that the groups Q^{a} and Q^{b} are selected from the group made up of: alcohols, aldehydes, primary amines, secondary amines, azides, ethynils, halogens, thiols, vinyls, and/or in that the spacer E is selected from the group made up of aromatics, heteroaromatics, saturated alkyl chains (branched or not), unsaturated alkyl chains (branched or not), glycols (and preferably polyethylene glycol).

3. The method according to claim 1, characterized in that said units Q^{a}-E-Q^{b} are selected from the group made up of: natural or unnatural or synthetic α, β, γ or δ-amino acids, natural or unnatural or synthetic α, β, γ or δ-hydroxy acids, natural or unnatural or synthetic α, β, γ or δ-mercapto acids.

4. The method according to claim 3, characterized in that said units of α, β or γ-amino acids or α, β or γ-hydroxy acids or α, β or γ-mercapto acids are used in an activated form.

5. The method according to claim 3, characterized in that said units of α, β or γ-amino acids or α, β or γ-hydroxy acids or α, β or γ-mercapto acids are implemented respectively in the form of 2,2-bis(trifluoromethyl)-1,3-oxazolidin-5-one or (2,2-bis(trifluoromethyl)-1,3-oxazinan-6-one or 2,2-bis(trifluoromethyl)-1,3-oxazepan-7-one or 2,2-bis(trifluoromethyl)-1,3-dioxolan-4-one or 2,2-bis(trifluoromethyl)-1,3-dioxan-4-one or 2,2-bis(trifluoromethyl)-1,3-dioxepan-4-one or 2,2-bis(trifluoromethyl)-1,3-oxathiolan-5-one or 2,2-bis(trifluoromethyl)-1,3-oxathian-6-one or 2,2-bis(trifluoromethyl)-1,3-oxathiepan-7-one or derivatives thereof.

6. The method according to claim 1, characterized in that said anchoring molecule includes a polyolefin chain or is a polyolefin chain or a polyolefin or polyalkene oligomer, with at least 10 monomer units, and preferably between 15 and 350 monomer units and is preferably a polyisobutene chain.

7. The method according to claim 6, characterized in that said polyolefin or polyolefin or polyalkene oligomer chain is functionalized at least at one of its ends.

8. The method according to claim 6, characterized in that said polyolefin or polyolefin or polyalkene oligomer chain comprises a number of unsaturated carbon-carbon bonds not exceeding 5%, and preferably not exceeding 3%.

9. The method according to claim 6, characterized in that said anchoring molecule has a weight average molecular weight comprised between 600 and 20000, and preferably between 700 and 15000.

10. The method according to claim 1, characterized in that said anchoring molecule includes a polyolefin chain (or is a polyolefin chain) or polyolefin or polyalkene oligomer terminated by at least one group selected from the group made up of:
a function —X^{a}, where X^{a} is selected from the group made up of: —OH, —NH₂, —NHR^{a}(R^{a}=alkyl or aryl), —SH;
a function —Y—C₆H₄X^{b}, where
Y is O, S, CH₂ or absent,
X^{b} is selected from the group made up of: —OH, —NH₂, —NHR^{a}, —SH, —CX^{a}R^{a}R^{b}, —C₆H₃R^{c}(CR^{a}X^{a}),
where R^{b} is selected from the group made up of —H, -Aryl, —Heteroaryl, -Alkyl, and R^{c} is selected from the group made up of —H, -Alkyl, —O-Alkyl, -Aryl, —O-Aryl, -Heteroaryl, —O-Heteroaryl;
a function —CR^{d}═CH—CHX^{a} or a function —CR^{d}H—CH═CH—CHX^{a}, where X^{a} has the meaning defined above, and R^{d} is methyl or ethyl.

11. The method according to claim 1, characterized in that:
said first end of said peptide or protein or peptidomimetic chain is a first unit of α, β or γ-amino acid or α, β or γ-hydroxy acid or α, β or γ-mercapto acid,
said peptide or protein or peptidomimetic chain is formed of n units of α, β or γ-amino acid and/or α, β or γ-hydroxy acid and/or α, β or γ-mercapto acid, and
the second end of said peptide chain is another unit of α, β or γ-amino acid or α, β or γ-hydroxy acid or α, β or γ-mercapto acid.

12. The method according to claim 1, characterized in that during said elongation another unit of α, β or γ-amino acid or α, β or γ-hydroxy acid or α, β or γ-mercapto acid is added to said second end from their respective activated forms.

13. The method according to claim 1, characterized in that said peptide or said protein or said peptidomimetic is obtained by condensation of a peptide or protein or peptidomimetic anchored on an anchoring molecule and of a fragment of peptide or protein or peptidomimetic suitably protected on its side chains.

14. The method according to claim 1, comprising at least one step wherein said peptide or protein or peptidomimetic chain attached to said anchoring molecule is separated from the reaction medium by extraction in an organic solvent (such as: cyclohexane, heptane, hexane(s)) by extraction or washing with water or a water/ethanol or water/acetonitrile mixture or by simple filtration.

15. The method according to claim 1, comprising a step wherein said peptide or said protein or said peptidomimetic is detached from said anchoring molecule.

16. A molecule that can be obtained by the method according to claim 1.


SEQUENCE LISTING (2 sequences)

SEQ ID NO: 1 - protein, 8 aa, Unknown
  This sequence is provided as an example in the pending application.
  PEPTIDE (1)..(8): Xaa3, Xaa5 and Xaa8 represent L-cysteine residues whose side chains are side chains protected by a benzyl group.
FWXWXWWX

SEQ ID NO: 2 - protein, 8 aa, Unknown
  This sequence is provided as an example in the pending application.
  peptide (1)..(8): Xaa1, Xaa4 et Xaa6 represent L-cysteine residues whose side chains are side chains protected by a benzyl group. Xaa8 represents a Phe residue modified with a polyisobutene derivative (PIB).
XWWXWXWX